(19) United States
(12) Patent Application Publication
(10) Pub. No.: US 2025/0222247 A1
(43) Pub. Date: Jul. 10, 2025

(54) IMPLANTABLE VENTRICULAR ASSIST SYSTEM AND METHOD FOR OPERATING SAME

(71) Applicant: KARDION GMBH, Stuttgart (DE)

(72) Inventor: Thomas Alexander Schlebusch, Renningen (DE)

(21) Appl. No.: 19/088,678

(22) Filed: Mar. 24, 2025

Related U.S. Application Data
(63) Continuation of application No. 15/734,342, filed on Jul. 30, 2021, now Pat. No. 12,257,424.

(30) Foreign Application Priority Data
Jun. 6, 2018 (DE) 102018208913.2

(51) Int. Cl. A61M 60/13; A61M 60/178; A61M 60/216; A61M 60/523; A61M 60/816

(52) U.S. Cl. CPC A61M 60/13; A61M 60/178; A61M 60/216; A61M 60/523; A61M 60/816; A61M 2205/3317; A61M 2205/3375

(57) ABSTRACT

The invention relates to a method for operating an implanted, ventricular assist system (2), comprising the following steps: a) determine a first impedance parameter at a first point in time by means of the assist system (2), b) determine a second impedance parameter at a second point in time by means of the assist system (2), c) at least determine a change of the impedance parameter using the first impedance parameter and the second impedance parameter, or compare at least the first or second impedance parameter to a threshold value.

### INCORPORATION BY REFERENCE TO ANY PRIORITY APPLICATIONS

The present application is a continuation of U.S. application Ser. No. 15/734,342 filed on Jul. 30, 2021, which is a National State entry of PCT Patent Application No. PCT/EP2019/064779, filed on Jun. 6, 2019, titled IMPLANTABLE VENTRICULAR ASSIST SYSTEM AND METHOD FOR OPERATING SAME, which is an International Application of and claims the benefit of priority to German Patent Application No. 102018208913.2, filed on Jun. 6, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention relates to a method for operating an implanted ventricular assist system, a processing unit and an implantable ventricular assist system. The invention is used in particular for (fully) implanted left heart assist systems (LVAD [Left Ventricular Assist Device]).

### Description of the Related Art

Implanted left heart assist systems (LVADs) mainly exist in two design variants. On the one hand, there are (percutaneous) minimally-invasive left heart assist systems. The second variant are left heart assist systems invasively implanted under the rib cage. The first variant circulates blood directly from the left ventricle into the aorta, since the (percutaneous) minimally invasive left heart assist system is positioned in the center of the aortic valve. The second variant circulates the blood from the apical region from the left ventricle via a bypass tube into the aorta.

The task of a cardiac assist system is to circulate blood. The so-called heart-time-volume (Herz-Zeit-Volumen [HZV], usually stated in liters per minute) has high clinical relevance in this case. In other words, the heart-time volume affects the total volume flow of blood from a ventricle, particularly from the left ventricle to the aorta. Accordingly, the initial task is to determine this parameter as a metrology value while a cardiac assist system is in operation.

Depending on the level of assistance, which describes the share of the volume flow from the ventricle to the aorta conveyed by a circulation means, such as a pump of the assist system, a certain volume flow reaches the aorta via the physiological path through the aortic valve. The heart-time volume or the total volume flow (Q_{HZV}) from the ventricle to the aorta is therefore usually the sum of the pump volume flow (Qₚ) and the aortic valve volume flow (Qₐ).

An established method for determining the heart-time volume (Q_{HTV}) in the clinical setting is the use of dilution methods, which, however, all rely on a catheter inserted transcutaneously and therefore can only provide heart-time volume measurement data during cardiac surgery. An established method for measuring the pump volume flow (Qₚ) is the correlation of the operating parameters of the assist system, predominantly the electrical power consumption, possibly supplemented by further physiological parameters, such as the blood pressure. The integration of dedicated ultrasound measurement technology into an assist system has also already been proposed.

### SUMMARY

A (fully) implanted recording of the heart-time volume, i.e., of Q_{HZV}, in particular by the assist system itself, has not yet been suggested or implemented. Fully implanted in this case means, in particular, that the means required for recording are completely in the patient's body and remain there. This makes it possible to record the heart-time volume even outside of heart surgery.

Impedance cardiography is a method for determining the so-called cardiac output using extracorporeal impedance measurements. Four electrodes are used for this. A small alternating current is fed in by two electrodes; the resulting voltage drop is measured with two additional electrodes. Since blood has a higher conductivity than the surrounding tissue, especially compared to the air-filled lung, the blood volume change in the thorax can be detected as an impedance change over a cardiac cycle. Impedance cardiography is used extracorporeally, usually with ring or adhesive electrodes around the neck and abdomen.

The use of left ventricular impedance together with the electrocardiogram to detect abnormal mechanical contractions of the heart muscle in LVAD patients is also known. An intracardiac ECG is in this case combined with a left ventricular impedance measurement to detect abnormal contractions of the ventricle. The measurement is synchronized by the ECG. The measurement is qualitative, and volume is not determined.

The use of impedance measurements for quantitative volume determination is known in the area of the bladder volume. Four electrodes are used on the outside of a catheter, which is only inserted into the bladder for measuring purposes and then removed again. It has been shown that the measured impedance is inversely dependent on the volume of the bladder. However, a problem of the method is the strong dependency on the urine conductivity.

Based on this, the invention is based on the task to further improve a method for operating an implanted, ventricular assist system for recording special parameters and to specify a corresponding advantageous system.

To achieve this task, combinations of features described herein are proposed. Advantageous configurations and further developments of the invention are described herein.

According to one aspect of the invention, a method for operating an implanted, ventricular assist system comprises:
a) determining a first impedance parameter at a first point in time using the assist system,
b) determining a second impedance parameter at a second time point using the assist system,
c) determining at least a change in the impedance parameter using the first impedance parameter and the second impedance parameter, or compare at least the first or second impedance parameter to a threshold value.

The assist system preferably serves the purpose of circulating fluid. The ventricular assist system is preferably a cardiac assist system. The method preferably serves to determine a total fluid volume flow (through a cross-section in the region of the assist system) from a ventricle of a heart, in particular from a (left) ventricle of a heart to the aorta in the region of a (fully) implanted, (left) ventricular (heart) assist system, and/or to determine an aortic valve or bypass volume flow that flows past the assist system. The fluid is regularly blood. The assist system is preferably arranged at the exit of the left ventricle of the heart or the left ventricle. The assist system is particularly preferably arranged in the aortic valve position. In particular, the total volume flow is defined as the total volume flow through a blood vessel or through a cross-section of the blood vessel. The blood vessel is, for example, the aorta, particularly in the case of a left heart assist system, or the pulmonary trunk (Truncus pulmonalis) into the two pulmonary arteries, particularly in the case of a right heart assist system. The method is particularly suitable for determining the total heart-time volume (HTV, with formula symbol Qhzv) of a patient, in particular with a (fully) implanted left ventricular heart assist system (LVAD) in the aortic valve position and/or through the assist system itself.

The method is based in particular on the integration of a ventricular impedance measurement, in particular impedance spectroscopy into a (left) ventricular (heart) assist system (LVAD), preferably for determining the heart-time volume (HTV) and/or the aortic valve or bypass volume flow. In addition, the procedure may contribute towards determining the level of assistance. The method advantageously makes it possible for the cardiac-time volume and/or the bypass volume flow to be provided outside the surgical scenario with a quality comparable to using a dilution catheter. This is particularly advantageous, since the heart-time volume (Q_{HZV}) has a greater clinical relevance than the more commonly used pump volume flow (Qₚ), which only quantifies the flow through the assist system itself. The solution proposed here is characterized in particular by the use of sensors integrated strictly in the LVAD; for example, no separate ultrasonic cuff is required around the aorta.

In step a), an (at least) first impedance parameter is determined at a first point in time by means of the assist system. A first ventricular volume is preferably determined at a first point in time in step a) by means of an impedance measurement by means of the assist system or by means of the first impedance parameter. In other words, this means in particular that the assist system itself determines, in particular measures, the impedance parameter or the ventricular volume. In step b), an (at least) second impedance parameter is determined at a second point in time by means of the assist system. A second ventricular volume is preferably determined in step b) at a second point in time by means of an impedance measurement by means of the assist system. In other words, this means in particular that in steps a) and b) at least one impedance parameter and/or one ventricular volume is determined over time. The first and second points in time differ from each other. Preferably, the second point in time is after the first point in time. A plurality of impedance parameters or ventricular volumes can be determined at other points in time. Short gaps between the points in time are particularly advantageous for forming a differential to determine the volume flow. The ventricular volume is limited in particular by the ventricular wall or the ventricular chamber wall formed in the manner of a bag and by two heart valves, one of which is also called an aortic valve. The ventricular volume is essentially determined by the fluid volume (here, in particular, blood volume) in the ventricle. The differences in ventricular volume result in particular from the contraction of the heart muscle and usually contribute to the circulation of blood by the cardiovascular system of humans, or patients if applicable.

The impedance parameter can for example be a (bio) impedance, in particular of fluid and/or tissue in the region of the assist system. Raw data of an analog/digital transducer (unitless values) of an impedance measurement device can also serve as impedance parameters. The impedance parameter is preferably a ventricular impedance. In other words, this means in particular that a ventricular impedance is determined in steps a) and b). Ventricular impedance is regularly a function of ventricular volume, in particular the fluid volume in the ventricle, and fluid conductivity (in particular blood conductivity). In addition, the muscles surrounding the ventricle can also provide an impedance component. The influence of the blood conductivity can be determined by separate impedance measurement in a known sample volume. In particular, a measurement in the defined volume of the (inlet) cannula is a candidate in this case. The impedance measurement serves in particular to determine the pulsatile volume change of the ventricle during the systole. In an advantageous embodiment, the impedance data can be validated by means of pressure sensor data.

It is particularly preferred that the impedance of the ventricle (of unknown volume) is determined by electrodes integrated into and/or in the assist system. The electrodes (for ventricular impedance measurement) are preferably arranged on or in the surface, in particular on or in the outer surface of the assist system (circumferentially). The electrodes are particularly preferably arranged on or in the outer surface of a(n) (inlet) cannula of the assist system (circumferentially).

In step c), a change (over time) of the impedance parameter is determined using the first impedance parameter and the second impedance parameter. Step c) can alternatively or cumulatively compare at least the first or second impedance parameter to a threshold value. A change (over time) of a ventricular impedance is preferably determined using the first impedance parameter and the second impedance parameter. The threshold value is preferably a (pre-)defined and/or constant threshold value. The threshold value can for example be determined, in particular specified, with a calibration (in vivo) as a particularly lower threshold for the impedance parameter or corresponding raw data values. The lower threshold is dimensioned in particular such that a collapse of the ventricle and/or a suction effect, which leads to a suctioning of the assist system to the inner ventricle wall, can be avoided. Step c) preferably determines a change (over time) in the ventricular volume using the first impedance parameter and/or the first ventricular volume and the second impedance parameter and/or the second ventricular volume. Step c) can alternatively or cumulatively compare the first and/or second impedance parameter and/or ventricular volume to a minimum ventricular volume. The change of the impedance parameter or ventricular volume is in particular determined if the assist system is implanted in a patient with partial assistance (low to high assistance level). The comparison to the threshold or the minimum ventricular volume is in particular made if the assist system is implanted in a patient with full assistance (assistance level=100%). If only one comparison with the threshold or minimum ventricular volume is to be performed, one of steps a) or b) can be omitted. In addition, the determination of the change and the comparison can also be performed cumulatively, in particular simultaneously, or at least partially in parallel, for example in a recovering short-term (full) assistance patient. Alternatively or cumulatively to the lower threshold or minimum ventricular volume, an upper threshold value or maximum ventricular volume can be specified for the comparison. The upper threshold value and/or the minimum ventricular volume is dimensioned in particular such that an excessive load, in particular an expansion of the ventricular wall, can be avoided.

In patients with partial assistance, the change in the impedance parameter or the ventricular volume during the systole (contraction or blood outflow phase of the heart) can contribute toward transporting blood through the cardiovascular system, or in other words contribute toward maintaining or increasing the total fluid volume flow from the ventricle or the heart-time volume. In these patients in particular, the determination of the change of the impedance parameter or ventricular volume is particularly important. The pulsatile impedance parameters or volume change of the ventricle is preferably determined during the systole. In other words, this means in particular that in step c) the (pulse or muscle-induced) change in the impedance parameter or ventricular volume is preferably determined during the systole. Particularly preferably, the difference between the impedance parameter or ventricular volume is determined at the start of the systole, and between the impedance parameter or ventricular volume at the end of the systole. For this purpose, the first point in time can for example be the start of the systole and the second point in time the end of the systole. In this exemplary illustration, however, it should be taken into account that the sampling rate of the impedance parameter or ventricular volume should be high enough to fulfill the Nyquist theorem for expected ventricular contractions, for example 60 l/s.

In patients with full assistance (especially with a continuously operational assist system), the change in impedance parameter or ventricular volume plays a rather subordinate role in particular. Rather, care should be taken in these patients to ensure that the impedance parameter or the ventricular volume does not fall below a lower threshold value or a minimum ventricular volume. The minimum ventricular volume is preferably a defined or predetermined and/or constant minimum ventricular volume. The minimum ventricular volume is dimensioned in particular such that a collapse of the ventricle and/or a suction effect, which leads to a suctioning of the assist system to the inner ventricle wall, can be avoided. The comparison can be performed in the processing unit. For this purpose, the measuring device can provide the (first and/or second) impedance parameter or the (first and/or second) ventricular volume as the initial value of the processing unit. The impedance parameter or the ventricular volume can represent a control parameter for the assist system, which is preferably held above the lower threshold value or the minimum ventricular volume. In addition, an impedance parameter to threshold or ventricular volume to minimum ventricular volume difference can represent a control parameter for the assist system. A processing unit of the assist system can provide this control parameter as an output parameter, in particular of a control unit of the assist system, which preferably regulates the output of an electric motor of the assist system and thus also in particular the (blood) circulation volume of the assist system. For example, the circulation volume of the assist system can be reduced if there is a risk of suctioning of the ventricular wall. In addition, the circulation volume of the assist system can be increased, for example, if there is a risk of excessive expansion of the ventricular wall.

In particular, if a plurality of impedance parameters or ventricular volumes are determined in steps a) and b), it is particularly advantageous that at least one model graph is adjusted to the impedance parameters or ventricular volumes. It is then also preferred if processing continues with the model parameters or the at least one model graph.

According to an advantageous embodiment, it is proposed that a ventricular volume is determined with the (respective) impedance parameter. In other words, this means in particular that an impedance measurement is carried out to determine the ventricular volume or that a ventricular volume is determined from the impedance measurement. Preferably, the first impedance parameter is used to determine a first ventricular volume, and the second impedance parameter is used to determine a second ventricular volume.

According to a further advantageous embodiment, it is proposed that the (respective) impedance parameters are measured by at least two electrodes arranged on the assist system. The first and the second impedance parameters (the respective ventricular impedance) are particularly preferably measured by at least two, advantageously four electrodes. For this purpose, a (smaller) patient auxiliary current, for example 50 kHz, 100 μA (alternating current) can be used for measurement. The electrodes are also preferably spaced apart from one another such that their detection range or detection volume comprises the ventricular volume. The detection range or the detection volume relates in particular to the region or volume that is covered by the current paths between the electrodes. In other words, this relates in particular to the region or the volume through which or such that the current paths extend between the electrodes. The at least two electrodes preferably comprise at least two electrode pairs, in particular each comprising a current electrode and a voltage measurement electrode. The electrodes of a pair should lie close to each other. However, the pairs should be separated from each other. An arrangement is advantageous wherein the current electrodes are located outside and the voltage measuring electrodes are located inside (in the form of a four-wire measurement). The detection range or the detection volume is generally increased with increasing distance between the electrode pairs. The distance is preferably determined such that the entire volume of the ventricle can be detected as far as possible, but the surrounding tissue and/or surrounding organs, in particular the lungs, are not in the detection range as far as possible.

According to a further advantageous embodiment, it is proposed that an impedance measurement is carried out at different (alternating current) frequencies, in particular for determining the first and/or second impedance parameters. This can advantageously contribute toward reducing the influence of surrounding tissue, in particular the heart muscles, on the impedance measurement. Particularly preferably, a (bio) impedance spectroscopy is performed. Preferably, the frequencies are selected such that the background impedance of the heart muscle can be or is determined, particularly preferably periodically. This background impedance can be taken into account for the impedance measurement of the ventricular volume.

According to an advantageous embodiment, it is proposed that the conductivity of the fluid be determined by an impedance measurement in a defined volume of the assist system. This contributes in particular to the fact that the assist system itself can determine the conductivity of the fluid, which has an influence on the impedance measurement. The defined volume is preferably located inside a(n) (inlet) cannula of the assist system. The defined volume is particularly preferably limited by the inner surface or a part of the inner surface of the cannula of the assist system. In addition, the defined volume (in a direction along the cannula) can be limited by two electrodes. Preferably, at least one of the (limiting) electrodes is in this case arranged on or in the inner surface of the cannula. Furthermore, it is preferred that at least two, advantageously four, electrodes are arranged for conductivity measurement on or in the inner surface of the cannula (circumferentially).

According to an advantageous embodiment, it is proposed that a fluid volume flow that flows through the assist system is also determined. In other words, this relates in particular to a fluid volume flow that only flows through the assist system itself. The fluid volume flow is usually the so-called pump volume flow (Qₚ), which only quantifies the flow through the support system itself. If this value is known in addition to the total volume flow or heart-time volume (Qhzv), the so-called level of assistance can be calculated from the ratio of Q_{P} to Q_{HZV} (i.e., Q_{P}/Q_{HZV}). To determine the pump volume flow, an established method discussed above in connection with the prior art can be used for measuring the pump volume flow. For example, the pump volume flow can be determined based on differential pressure and motor characteristic field or by explicit Doppler ultrasonic measurement. The pump volume flow is preferably determined by means of an ultrasonic sensor, which can for example be accommodated in a tip of the assist system. The fluid volume flow graph versus time or the pump volume flow is preferably determined. The latter can be compared to total fluid volume graph over time. The level of assistance can be provided as a control parameter for the assist system.

According to an advantageous embodiment, it is proposed that a total fluid volume flow be determined in the region of the assist system. The total fluid volume flow is preferably determined using the first ventricular volume and the second ventricular volume. In cases in which the total fluid volume flow corresponds essentially to the time-related derivative of the ventricular volume, the total fluid volume flow can be (approximately) determined in the form of a difference ratio, which is the ratio (ΔV/Δt) of the ventricular volume difference (ΔV=second ventricular volume−first ventricular volume) to the time difference (Δt=second point in time (e.g., systole end)−first point in time (e.g., systole start). Cases in which the total fluid volume flow essentially corresponds to the time-related derivative of the ventricular volume can in particular occur with low assistance levels and/or pulsatile operating assist systems, for example controlled by a pressure sensor in the ventricle or an ECG sensor. In addition, an estimate of the total heart-time volume (HZV) can also be made using the measured pump volume flow and the measured ventricular volume. Preferably, the total fluid volume flow or the heart-time volume is calculated by sampling the ventricular volume and the fluid volume flow rate or pump volume flow versus time. The total fluid volume flow or the heart-time volume (Qhzv) can generally (at least for continuously operating assist systems) be determined from the following relationship between change of the ventricular volume (∂V/∂t) versus time and the basic pump volume flow (Q_{p,diastole}):

$${Q}_{H Z V} ( t ) = {Q}_{p} , Diastole - \frac{\partial V}{\partial t}$$

The determined total fluid volume flow is preferably provided as a control parameter for the assist system. A processing unit of the assist system can provide this control parameter as an output parameter, in particular of a control unit of the assist system, which preferably regulates the output of an electric motor of the assist system and thus in particular also regulates the (blood) circulation volume of the assist system.

Furthermore, an aortic valve or bypass volume flow (Q_{A}) is preferably determined. Particularly preferably, the bypass flow (aortic valve or bypass volume flow) is quantified as a bypass to the assist system and through the aortic valve by means of the ventricular volume change and pump volume flow:

$${Q}_{A} ( t ) = - \frac{\partial V}{\partial t} + {Q}_{p} , Diastole - {Q}_{p} ( t )$$

The bypass volume flow can be provided as a control parameter for the assist system.

According to a further aspect, a processing unit is proposed that is configured for carrying out a method proposed here. The processing unit can comprise a microprocessor, which can access a memory. The processing unit preferably receives data from the measuring device.

According to a further aspect of the invention, an implantable, ventricular assist system is proposed, comprising:
a measuring device, configured for determining a first impedance parameter at a first point in time and a second impedance parameter at a second point in time,
a processing unit, configured at least to determine a change of the impedance parameter (over time) using the first impedance parameter and the second impedance parameter or to compare at least the first or second impedance parameter to a threshold value.

The assist system is preferably a left-ventricular heart assist system (LVAD) or a percutaneous, minimally invasive left heart assist system. Furthermore, it is preferred that the assist system can be fully implanted. In other words, this means, in particular, that the means required for sensing, in particular an impedance sensor and/or the pump volume flow sensor, are completely located in the body of the patient and remain there. The assist system is particularly preferably configured and/or suitable for being arranged at least partially in a ventricle, preferably in the left ventricle of a heart, and/or in an aorta, in particular in the aortic valve position.

Furthermore, the assist system preferably comprises a cannula, in particular an inlet cannula, a flow machine, such as a pump and/or an electric motor. The electric motor is regularly a component of the flow machine. The (inlet) cannula is preferably arranged such that in the implanted state it can guide fluid from a (left) ventricle of a heart to the flow machine. The assist system is preferably elongated and/or tubular. Preferably, the inlet cannula and the flow machine are arranged in the region at opposite ends of the assist system.

According to an advantageous embodiment, it is proposed that the measuring device comprises at least two electrodes by which an impedance can be measured. At least four electrodes are preferably provided for the measurement of a ventricular impedance. Particularly preferably, the electrodes are configured and arranged such that the impedance of the (entire) ventricle can be determined.

The details, features, and advantageous embodiments discussed in connection with the method can also occur accordingly in the processing unit and/or the assist system presented here and vice versa. In this respect, reference is made in full to the statements therein regarding the more detailed characterization of the features.

### BRIEF DESCRIPTION OF THE DRAWINGS

The solution presented here as well as its technical environment are explained in more detail below with reference to the figures. It should be pointed out that the invention is not to be limited by the exemplary embodiments shown. In particular, unless explicitly stated otherwise, it is also possible to extract partial aspects of the facts explained in the figures and to combine them with other components and/or insights from other figures and/or the present description. The following show schematically:

FIG. 1 an implantable assist system,

FIG. 2 an implanted ventricular assist system in a heart,

FIG. 3 the assist system from FIG. 2,

FIG. 4 an implanted ventricular assist system, and

FIG. 5 a schematic process diagram of the described method.

### DETAILED DESCRIPTION OF THE PREFERRED EMBODIMENT

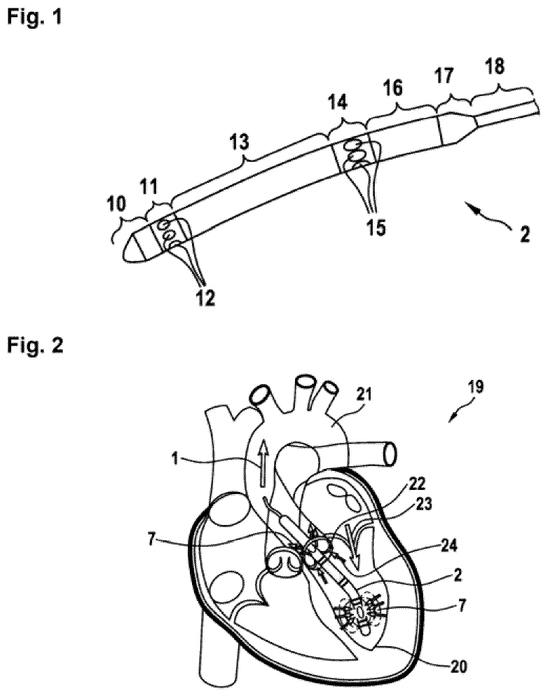

FIG. 1 schematically shows an implantable assist system 2. The assist system 2 is in this case an example of a left ventricular assist system (LVAD). The assist system 2 comprises a tip 10, which can contain sensors, an inlet cage 11 with feed openings 12 for suctioning fluid (here: blood), a flexible cannula 13, an impeller cage 14 with turbine wheel (not shown here) and outlet openings 15 for the blood, an electric motor 16, a rear end 17 (so-called backend), which can contain sensors, and a connection cable 18.

FIG. 2 shows schematically an implanted ventricular assist system 2 in a heart 19. The assist system 2 assists the heart 19 by assisting the circulation of blood from the (left) ventricle 20 into the aorta 21. For this purpose, the assist system 2 is anchored in the aortic valve 22, as illustrated in FIG. 2. At an assistance level of 100%, the assist system 2 (LVAD) circulates the complete blood volume flow. The level of assistance describes the proportion of the volume flow circulated by a conveying means, such as a pump of the assist system 2 or through the assist system 2, to the total volume flow of blood from the ventricle 20 to the aorta 21.

At an assistance level of 100%, the total fluid volume flow 1 from the ventricle 20, the heart valve volume flow 23 into the ventricle 20, and the fluid volume flow 7 through the assist system 2 are therefore identical. The aortic valve or bypass volume flow 24 (formula symbol: Qₐ) is therefore zero. The total fluid volume flow 1 can also be described as (total) heart-time volume (HZV, formula symbol: Q_{HZV}). The fluid volume flow 7 can also be referred to as a so-called pump volume flow (formula symbol: Qₚ), which only quantifies the flow through the assist system itself. The level of assistance can thus be calculated from the ratio Q_{P}/Q_{HZV}.

For lower levels of assistance and healthier hearts with strong ventricular contraction, the heart 19 continues to fulfill its function to a certain extent, so that a pulsatile volume flow fraction 24 (bypass) through the heart or aortic valve 22 is generated during the systole (heart muscle contracts and by reducing the volume of the ventricle 20 displaces blood into the aorta 21). At the same time, the pressure difference over the assist system 2 decreases, in particular over the normally provided pump (not shown here) of the assist system 2, so that the assist system 2 accordingly also conveys an increased fluid volume flow 7 during the systole.

The solution proposed here is based in particular on detecting the change of the ventricular volume over time by way of an impedance measurement. For a known (or recorded over time) fluid volume flow 7 or pump volume flow (Qₚ), the otherwise barely measurable aortic valve or bypass volume flow 24 can then be advantageously quantified by determining the differential.

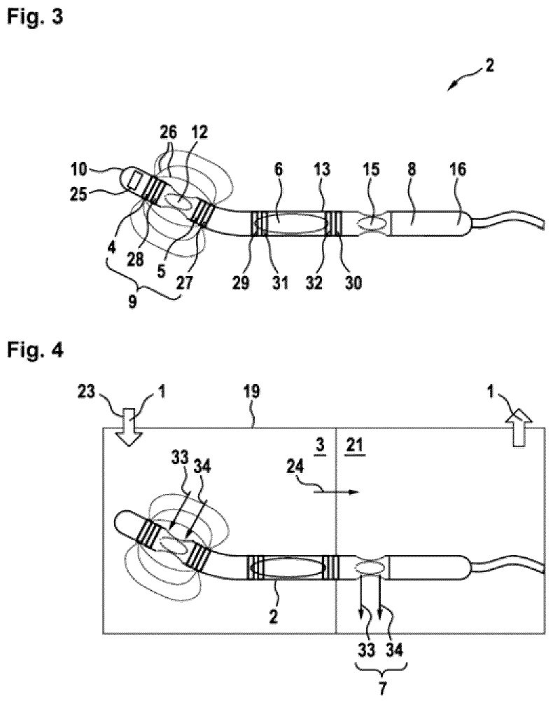

FIG. 3 schematically shows the assist system 2 from FIG. 2. To determine the ventricular volume at a specific point in time, FIGS. 2 and 3 show a realization form by way of a (left ventricular) intracardiac impedance measurement. The impedance measurement has the advantage that the entire fluid or liquid volume and an electrical resistance measurement can be implemented with relatively straightforward technical means. By using four electrodes 4, 5, 27 and 28 (four-wire measurement) as in the example shown here, the impedance measurement is largely independent of the contact resistance of the measurement electrodes, so that the measurement result is also suitable for long-term implantation in the patient. Electrodes 4, 5, 27 and 28 in this case form an exemplary measuring device 9 of the assist system 2. FIG. 3 also illustrates by way of example that an ultrasonic-based pump volume flow sensor 25 can be integrated in the tip 10. The pump volume flow sensor 25 can detect and supply the fluid volume flow 7 and/or the pump volume flow (Qₚ) over time.

For the impedance measurement, at least two electrodes 4, 27 should be integrated in the region of the ventricle 20, so that the current paths 26 of the measuring current can record the ventricular volume as best as possible. The region of the inlet cage 11 is suited for this purpose, for example proximally and/or upstream and distally and/or downstream of the inlet openings 12. At least two electrodes 4, 27 are technically required to close the electrical circuit. The use of four electrodes 4, 5, 27 and 28 is advantageous, since the influence of the contact impedance from the electrode to the blood can be ignored; long-term changes of the electrode surface therefore have no or only a negligible influence on the measurement result. For example, the current electrodes 4, 27 are preferably placed on the outside and the voltage measurement electrodes 5, 28 between them. The current paths 26 in this case extend from the electrode 4 to the electrode 27 (current electrodes). Equipotential lines that are not shown here form between them, which can be measured at high resistance by the voltage electrodes 28 and 5.

Since the detection volume is dependent in particular on the distance of the measurement pairs (4 and 28 to 5 and 27), it is advantageous to place one pair 4, 28 as distally as possible or in the region of the tip 10 and one pair 5, 27 as proximally as possible or towards the electric motor 16 of the assist system 2. It is advantageous in this case for the measurement quality if all four electrodes 4, 5, 27 and 28 are nevertheless placed in the area of the ventricle 20 and the proximal pair 5, 27 does not move beyond the aortic valve plane into the aorta 21, wherein a measurement would also be conceivable or feasible based on this design.

The measured electrical conductivity between the electrode pairs is a function of the surrounding fluid or fluid volume and its conductivity. A predetermined and/or constant conductivity can be assumed, since the ion concentration in the blood is kept within narrow limits by the kidneys. Nevertheless, the explicit determination of blood conductivity is particularly preferred here. For this purpose, a defined volume 6 is required, as is for example present in the interior of the (inlet) cannula 13. Accordingly, a further one to four electrodes 29, 30, 31 and 32 can be placed inside the cannula 13 (which can also be referred to as the inlet tube). Analogous to the measurement in the ventricular volume, the measurement can in this case be made by current injection and voltage measurement in electrode pairs 29 and 30, or by current injection between the electrode pair 29 and 30 and voltage measurement between the electrode pair 31 and 32. If at least one electrode 4, 5, 27 or 28 of the ventricle measurement is located in the region or near the inlet opening 12, the pair 29, 31 can be omitted. The measurement can then for example be carried out by means of a current injection in the electrode pair 27, 30 and by a voltage measurement between the electrode pair 5 and 32. Since the electrode pair 5, 27 is located in the region or near the inlet opening 12, a defined volume 6 results inside the cannula 13 between the electrode pair 5, 27 and the electrode pair 30, 32. For strictly two-point measurements, three electrodes are therefore in particular minimally necessary. The ventricular impedance measurement is then carried out by way of example between electrodes 4 and 5, and the conductivity measurement between electrodes 5 and 30.

The assignment of the electrodes shown here as current electrodes or voltage measurement electrodes is only exemplary. A current electrode in this case is in particular an electrode that can be connected or is connected to a current source. A voltage measuring electrode is in particular an electrode that can be connected or is connected to a voltage measuring device. Any assignment that can fulfill the purpose of impedance measurement (of the ventricular volume and/or conductivity) presented here is possible. The voltage source and/or the voltmeter can be part of the measuring device 9. If the current source and the voltmeter are spatially separated from the assist system 2 by an electrical supply line 18, the supply line cable particularly advantageously has a triaxial design with active shielding.

For the measurement (of the ventricular volume and/or the conductivity), alternating current can for example be injected by a current electrode, and the resulting voltage drop can for example be measured by a voltage measurement electrode. However, voltage can also be applied and the resulting current flow can be measured.

The measurement can be carried out at a single (alternating current) frequency. Values in the range of 50 kHz have been established for bioelectrical impedance analysis and impedance tomography. In addition to blood volume, the surrounding heart muscles also contribute slightly towards the measured impedance. Since this is cellular material with a structure different from the cellular material in the blood, the influence can be reduced by way of the so-called bioelectrical impedance spectroscopy. The measurement is in this case not performed at a fixed frequency, but at several frequencies. The result is the electrical impedance as a function of the frequency (cf. dispersion, Cole chart).

For example, the measurement can be carried out in the range from 1 kHz to 1 MHz. The spectroscopic measurement can for example be carried out by a sequence of sine frequencies, a so-called chirp, or a broadband binary sequence (pseudo random noise).

The ventricular impedance sampling rate should be high enough to fulfill the Nyquist theorem for expected ventricular contraction frequencies, for example 60 l/s. In contrast to a volume change of the ventricle, impedance changes of the heart muscles are only expected very slowly, so that a complete frequency range sweep is not required for every measurement. The background impedance of the heart muscle can be determined periodically. It is also advantageous to determine the frequency measurement points of the background impedance over several heartbeats. For this purpose, the measurement can be carried out at two frequencies with a high sampling rate. The first frequency is preferably fixed, the second frequency changes, for example from heartbeat to heartbeat (e.g., determined based on the impedance plot of the first frequency). The spectrum of the separate measurements is combined by way of example using the impedance plot of the first frequency. Instead of the first frequency, synchronization is for example also possible based on the pressure plot of a pressure sensor.

FIG. 4 shows schematically an implanted ventricular assist system 2. FIG. 4 illustrates the resulting volume flows in the heart 19. The left volume represents the ventricle or the ventricular volume 3, the right volume the aorta 21. Due to mass conservation, the (heart valve) volume flow 23 from the left atrium into the ventricle and the outflow into the aorta corresponds to the total fluid volume flow 1 or the (total) heart-time volume. Valve insufficiency or hydraulic short circuits between the ventricles are exceptions.

The assist system 2 (LVAD) conveys the circulation volume flow 33, for example by the output of a pump (not shown here) of the assist system 2. In continuous assist systems, the flow is constant; in pulsatile assist systems, this flow is time-modulated. The contraction of the ventricle causes the pressure in the ventricle to increase and the pressure difference over the assist system 2 decreases, so that the assist system 2 delivers an additional systolic volume flow 34 at constant mechanical output during the systole. If the ventricular contraction is strong enough (e.g., for short-term assistance patients), the ventricular pressure can exceed the aortic blood pressure, which leads to the opening of the aortic valve (not shown here). An additional bypass flow (formula symbol Qₐ) is formed, which is shown in FIG. 4 as an aortic valve or bypass volume flow 24. This flow portion (bypass 24) cannot be (directly) detected by the flow sensors of the assist system 2, for example the ultrasonic sensor in the tip of the assist system 2, but essentially corresponds to the difference between the change in ventricular volume dV/dt and the pump volume flow Qₚ. In this case, the pump volume flow Qₚ, which only quantifies the flow through the assist system 2 itself, and which is also referred to here as fluid volume flow 7, results from the sum of the circulation volume flow 33 and the systolic volume flow 34. By combining the ventricular volume measurement over time and pump flow measurement over time, the bypass flow, i.e., the aortic valve or bypass volume flow 24 (formula symbol Qa) can be determined according to the following equation:

$${Q}_{A} ( t ) = - \frac{\partial V}{\partial t} + {Q}_{p} , Diastole - {Q}_{p} ( t )$$

Wherein the base pump volume flow Q_{p,diastole} (Qₚ during the diastole) in this case corresponds to the circulation volume flow 33, and the time-dependent pump volume flow Q_{P}(t) corresponds to the sum of the circulation volume flow 33 and the systolic volume flow 34. Q_{A}(t) corresponds to the aortic valve or bypass volume flow 24.

An exemplary derivation of this relationship as well as the heart-time volume is illustrated using the following equations 1 to 13. The formula symbols used are briefly explained first.
Q_{HZV} Heart time volume
Q_{P} Time-dependent volume flow through the pump
Q_{A} Time-dependent volume flow through the aortic valve
Q_{V} Time-dependent volume flow into the storage volume of the ventricle (ventricular volume change)
Q_{D} Time-dependent volume flow from the left atrium into the aorta (assumption: base flow through the pump)
V Ventricular volume
t time

$${Q}_{H Z V} = {Q}_{A} + {Q}_{P} \qquad ( 1 )$$

$${Q}_{H Z V} = {Q}_{D} + {Q}_{V} \qquad ( 2 )$$

$${Q}_{V} = - \frac{\partial V}{\partial t} \qquad ( 3 )$$

$${Q}_{A} + {Q}_{P} = {Q}_{D} - \frac{\partial V}{\partial t} \qquad ( 4 )$$

Assumption: Q_{D} flows exclusively through the pump (non-pulse equivalent part of the flow). During the systole, the pulsatile portion of the flow is divided into an increased Q_{P} and a bypass flow through the aortic valve Q_{A}:

$${Q}_{P} = {Q}_{D} + k \cdot - \frac{\partial V}{\partial t} \qquad ( 5 )$$

$${Q}_{A} = ( 1 - k ) \cdot - \frac{\partial V}{\partial t} \qquad ( 6 )$$

$$0 \leq k \leq 1 \qquad ( 7 )$$

The following is known from the (bio) impedance measurement:

$${Q}_{V} ( t ) = - \frac{\partial V}{\partial t} \qquad ( 8 )$$

The following is known from an exemplary ultrasound measurement:

$${Q}_{P} ( t ) = {Q}_{D} + k ( t ) \times - \frac{\partial V}{\partial t} \qquad ( 9 )$$

In addition, it can be assumed that the following applies during the diastole:

$${Q}_{P , Diastole} = {Q}_{D} \qquad ( 10 )$$

The bypass flow Q_{A} can thus be determined by:

$${Q}_{A} ( t ) = {Q}_{V} ( t ) + {Q}_{P , Diastole} - {Q}_{p} ( t ) \qquad ( 11 )$$

$$and$$

$${Q}_{A} ( t ) = - \frac{\partial V}{\partial t} + {Q}_{P , Diastole} - {Q}_{p} ( t ) \qquad ( 12 )$$

In addition, the heart-time volume Q_{hzv can be} determined by:

$${Q}_{H Z V} ( t ) = {Q}_{P , Diastole} - \frac{\partial V}{\partial t} \qquad ( 13 )$$

In addition, it is also preferred to place the assist system in a specific volume measurement mode. The pump is in this case regulated by integrated pressure sensors such that the pressure at the inlet cage and the impeller cage is identical, i.e., there is no volume flow and the pump rotates only so fast that there is no return flow from the aorta through the pump into the ventricle. At the same time, the beat volume, the final diastolic blood volume and/or the contraction strength of the ventricle can be determined based on the impedance measurement data (measured therein) graph over time, and/or used to control the assist system.

In addition to volume flow, the ventricular volume 3 is also an interesting parameter. In full assistance systems (only the pump circulates blood), a suction effect or collapse of the ventricle can occur. The assist system 2 in this case circulates more blood than flows in from the left atrium. The assist system 2 (or its pump) empties the ventricle. As a result, the ventricular wall approaches the inlet cage and the assist system 2 (or its pump) can suction itself against [the ventricular wall]. If the ventricular blood pressure falls below a value of 50 mmHg, the ventricle may also collapse due to higher ambient pressures. The ventricular volume 3 can in this case be used as a control parameter in order to reduce the output of the assist system 2, in particular by reducing its circulation or pumping output, so that a minimum ventricular volume can be guaranteed.

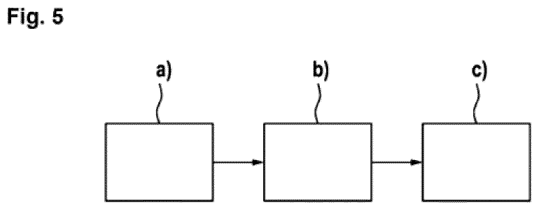

FIG. 5 shows the described method again in schematical form. The method steps a) determine a first impedance parameter at a first point in time by means of the assist system 2, b) determine a second impedance parameter at a second point in time by means of the assist system 2 and c) at least determine a change of the impedance parameter using the first impedance parameter and the second impedance parameter or compare at least the first or second impedance parameter to a threshold value are carried out in succession.

The solution proposed here in particular enables one or more of the following advantages:
Use of sensors strictly integrated in the assist system; for example, no separate ultrasound cuff is required around the aorta.
The method enables the determination of the total heart-time volume as well as the determination of the level of assistance (proportion of the pump volume flow of the total heart-time volume).
The integration of electrical impedance measurement technology is easy to implement compared to aortic ultrasonic cuffs or similar.
The total heart-time volume is measured continuously by the system, allowing the LVAD to be regulated both based on the heart-time volume as well as on the level of assistance, which is particularly advantageous in short-term assist systems (keyword weaning).
The method determines the ventricular volume, so that an assist system can be throttled, for example when a minimum ventricular volume is not reached.

What is claimed is:

1. A method for operating an implantable cardiac assist system, the method comprising:
measuring an impedance parameter using one or more electrodes of the implantable cardiac assist system, the implantable cardiac assist system comprising a cannula configured to supply a fluid, and a flow machine configured to generate a fluid flow;
determining, by a processor of the implantable cardiac assist system, an impedance parameter value of the impedance parameter at a first point in time measured by the one or more electrodes; and
determining, by the processor, if the impedance parameter value is below a threshold value.

2. The method of claim 1, further comprising determining a ventricular volume based at least in part on the impedance parameter.

3. The method of claim 1, further comprising throttling the implantable cardiac assist system when the impedance parameter exceeds the threshold value.

4. The method of claim 1, wherein the impedance parameter value is determined by at least two electrodes arranged on the implantable cardiac assist system.

5. The method of claim 1, further comprising determining fluid conductivity based at least in part on the impedance parameter value.

6. The method of claim 5, wherein determining the impedance parameter value and is performed in a defined volume of the implantable cardiac assist system.

7. The method of claim 1, further comprising determining a fluid volume flow that flows through the implantable cardiac assist system.

8. An implantable cardiac assist system comprising:
a cannula configured to supply a fluid;
a flow machine configured to generate a fluid flow;
one or more electrodes configured to measure an impedance parameter; a processor; and
a computer-readable storage device storing therein computer-readable instructions that, when executed by the processor, cause the processor to:
receive an impedance parameter from the one or more electrodes;
determine an impedance parameter value of the impedance parameter at a first point in time; and
compare at least the impedance parameter value to a threshold value.

9. The system of claim 8, wherein the processor is further configured to determine a ventricular volume based at least in part on the impedance parameter.

10. The system of claim 8, wherein the processor is further configured to determine fluid conductivity based at least in part on the impedance parameter value.

11. The system of claim 10, wherein determining the impedance parameter value is performed in a defined volume of the implantable cardiac assist system.

12. The system of claim 8, wherein the one or more electrodes comprises a first pair of electrodes and a second pair of electrodes, wherein the first pair of electrodes comprises a first current electrode and a first voltage measurement electrode and the second pair of electrodes comprises a second current electrode and a second voltage measurement electrode.

13. The system of claim 8, wherein the one or more electrodes comprises a first pair of electrodes and a second pair of electrodes, wherein the first pair of electrodes are positioned in a distal region of the cannula.

14. The system of claim 13, wherein the second pair of electrodes are positioned in a proximal region of the cannula.

15. An implantable cardiac assist system comprising:
a cannula configured to supply a fluid;
a flow machine configured to generate a fluid flow;
at least two electrodes arranged circumferentially on or in an outer surface of the implantable cardiac assist system, the at least two electrodes configured to measure a ventricular impedance associated with the implantable cardiac assist system; and
a processing unit configured to determine an impedance parameter value of the ventricular impedance at a first point in time and to compare the impedance parameter value to a threshold value.

16. The system of claim 15, wherein the at least two electrodes are configured to determine the ventricular impedance at different frequencies.

17. The system of claim 15, wherein the at least two electrodes are configured to be positioned in an area of a ventricle of a heart when the implantable cardiac assist system is implanted.

18. The system of claim 15, further comprising a device for determining a fluid volume flow.

19. The system of claim 18, wherein the processing unit is configured to throttle the fluid flow of the flow machine when a ventricular volume drops below a predetermined threshold value.

20. The system of claim 18, wherein the device for determining the fluid volume flow is an ultrasonic sensor.
